(19) United States
(12) Patent Application Publication
(10) Pub. No.: US 2006/0040287 A1
(43) Pub. Date: Feb. 23, 2006

(54) Method and system for quantifying random errors and any spatial-intensity trends present in microarray data sets

(76) Inventors: John F. Corson, Mountain View, CA (US); Charles D. Troup, Livermore, CA (US); Eric M. Leproust, San Jose, CA (US); Glenda C. Delenstarr, Redwood City, CA (US); Jayati Ghosh, Sunnyvale, CA (US); Patrick J. Collins, San Francisco, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 11/139,896

(22) Filed: May 26, 2005

Related U.S. Application Data
(60) Provisional application No. 60/576,562, filed on Jun. 2, 2004.

(51) Int. Cl. C12Q 1/68; G06F 19/00

(52) U.S. Cl. 435/6; 702/20

(57) ABSTRACT

A method and system for quantify random errors, sequence-dependent trends, and spatial-intensity trends in one or more channels of microarray data sets. The method and system of one embodiment of the present invention is directed to a method for quantifying random errors, sequence-dependent trends, and spatial-intensity trends present in microarray data sets. An additive error equation is employed to quantify background noise present in feature intensities due to random errors, sequence-dependent trends, and spatial-intensity trends.

### RELATED APPLICATION

This application claims priority to U.S. Provisional Application No. 60/576,562, filed Jun. 2, 2004, under 35 U.S.C. 119(e), the entirety of which is incorporated herein by reference.

### BACKGROUND OF THE INVENTION

The present invention is related to microarrays. In order to facilitate discussion of the present invention, a general background for particular types of microarrays is provided below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques to analyze complex solutions of any type of molecule that can be optically or radiometrically detected and that can bind with high specificity to complementary molecules synthesized within, or bound to, discrete features on the surface of a microarray. Because microarrays are widely used for analysis of nucleic acid samples, the following background information on microarrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. Phosphorylated subunits of DNA and RNA molecules, called “nucleotides,” are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations A, T, C, and G for the nucleotide subunits that together compose the DNA polymer. For example, the oligomer 100 shown in FIG. 1 can be chemically represented as “ATCG.”

The DNA polymers that contain the organization information for living organisms occur in the nuclei of cells in pairs, forming double-stranded DNA helices. One polymer of the pair is laid out in a 5′ to 3′ direction, and the other polymer of the pair is laid out in a 3′ to 5′ direction, or, in other words, the two strands are anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

FIGS. 4-7 illustrate the principle of the microarray-based hybridization assay. A microarray (402 in FIG. 4) comprises a substrate upon which a regular pattern of features is prepared by various manufacturing processes. The microarray 402 in FIG. 4, and in subsequent FIGS. 5-7, has a grid-like 2-dimensional pattern of square features, such as feature 404 shown in the upper left-hand corner of the microarray. Each feature of the microarray contains a large number of identical oligonucleotides covalently bound to the surface of the feature. These bound oligonucleotides are known as probes. In general, chemically distinct probes are bound to the different features of a microarray, so that each feature corresponds to a particular nucleotide sequence.

Once a microarray has been prepared, the microarray may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the microarray. FIG. 5 shows a number of such target molecules 502-504 hybridized to complementary probes 505-507, which are in turn bound to the surface of the microarray 402. Targets, such as labeled DNA molecules 508 and 509, that do not contain nucleotide sequences complementary to any of the probes bound to the microarray surface do not hybridize to generate stable duplexes and, as a result, tend to remain in solution. The sample solution is then rinsed from the surface of the microarray, washing away any unbound-labeled DNA molecules. In other embodiments, unlabeled target sample is allowed to hybridize with the microarray first. Typically, such a target sample has been modified with a chemical moiety that will react with a second chemical moiety in subsequent steps. Then, either before or after a wash step, a solution containing the second chemical moiety bound to a label is reacted with the target on the microarray. After washing, the microarray is ready for analysis. Biotin and avidin represent an example of a pair of chemical moieties that can be utilized for such steps.

Finally, as shown in FIG. 6, the bound labeled DNA molecules are detected via optical or radiometric instrumental detection. Optical detection involves exciting labels of bound labeled DNA molecules with electromagnetic radiation of appropriate frequency and detecting fluorescent emissions from the labels, or detecting light emitted from chemiluminescent labels. When radioisotope labels are employed, radiometric detection can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of instrumental detection produce an analog or digital representation of the microarray as shown in FIG. 7, with features to which labeled target molecules are hybridized similar to 702 optically or digitally differentiated from those features to which no labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the microarray was exposed, of labeled DNA complementary to the oligonucleotide within the feature.

One, two, or more than two data subsets within a data set can be obtained from a single microarray by scanning or reading the microarray for one, two or more than two types of signals. Two or more data subsets can also be obtained by combining data from two different arrays. When optical detection is used to detect fluorescent or chemiluminescent emission from chromophore labels, a first set of signals, or data subset, may be generated by reading the microarray at a first optical wavelength, a second set of signals, or data subset, may be generated by reading the microarray at a second optical wavelength, and additional sets of signals may be generated by detection or reading the microarray at additional optical wavelengths. Different signals may be obtained from a microarray by radiometric detection of radioactive emissions at one, two, or more than two different energy levels. Target molecules may be labeled with either a first chromophore that emits light at a first wavelength, or a second chromophore that emits light at a second wavelength. Following hybridization, the microarray can be read at the first wavelength to detect target molecules, labeled with the first chromophore, hybridized to features of the microarray, and can then be read at the second wavelength to detect target molecules, labeled with the second chromophore, hybridized to the features of the microarray. In one common microarray system, the first chromophore emits light at a near infrared wavelength, and the second chromophore emits light at a yellow visible-light wavelength, although these two chromophores, and corresponding signals, are referred to as “red” and “green.” The data set obtained from reading the microarray at the red wavelength is referred to as the “red signal,” and the data set obtained from reading the microarray at the green wavelength is referred to as the “green signal.” While it is common to use one or two different chromophores, it is possible to use one, three, four, or more than four different chromophores and to read a microarray at one, three, four, or more than four wavelengths to produce one, three, four, or more than four data sets. With the use of quantum-dot dye particles, the emission is tunable by suitable engineering of the quantum-dot dye particles, and a fairly large set of such quantum-dot dye particles can be excited with a single-color, single-laser-based excitation.

Sources of background noise can inflate the signal intensities associated with certain of the features of the microarray. Manufacturers and designers of microarrays and microarray readers, as well as researchers and diagnosticians who use microarrays in experimental and commercial settings, have recognized the need for an accurate method and system for quantifying and removing background noise from microarray data sets.

### SUMMARY OF THE INVENTION

Various embodiments of the present invention are directed to detecting and removing background noise from measured signal intensities of microarray features that together compose a microarray data set. One of many possible embodiments of the present invention is directed toward a method and system for quantifying random errors, sequence-dependent trends, and spatial-intensity trends present in microarray data sets. An additive error equation is employed to quantify background noise present in feature intensities due to random errors, sequence-dependent trends, and spatial-intensity trends.

### BRIEF DESCRIPTION OF THE DRAWINGS

FIG. 1 illustrates a short DNA polymer.

FIGS. 2A-B illustrate the hydrogen bonding between the purine and pyrimidine bases of two anti-parallel DNA strands.

FIG. 3 illustrates a short section of a DNA double helix comprising a first strand and a second, anti-parallel strand.

FIG. 4 illustrates a grid-like, two-dimensional pattern of square features.

FIG. 5 shows a number of target molecules hybridized to complementary probes, which are in turn bound to the surface of the microarray.

FIG. 6 illustrates the bound labeled DNA molecules detected via optical or radiometric scanning.

FIG. 7 illustrates optical, radiometric, or other types of scanning produced by an analog or digital representation of the microarray.

FIG. 8 illustrates a hypothetical intensity versus DNA mass plot.

FIGS. 9A-B illustrate log ratio versus log magnitude plots for hypothetical dye-swap microarray hybridization assays.

FIGS. 10A-B show a contour plot of a spatial-intensity trend for a hypothetical microarray and a path through the contour plot.

FIGS. 11A-B show both a negative-control feature having a uniform intensity distribution and a negative-control feature having a non-uniform intensity distribution.

FIG. 12 illustrates a hypothetical distribution of a set of negative-control feature intensities.

FIG. 13 illustrates the concept of a p-value.

FIG. 14 is a control-flow diagram representing one of many possible methods for determining additive error constants for the universal error model, m₁ and m₂.

FIG. 15 is a plot of the percent of crossover genes versus additive error for a hypothetical, dye-swap microarray hybridization assay.

FIG. 16 is a plot of the additive error AddError versus Target_A values for six different hypothetical microarray data sets.

FIG. 17 is a plot of intensity versus percentage of adenosine monomer present in a number of hypothetical negative-control oligonucleotide probes having different nucleotide sequence compositions.

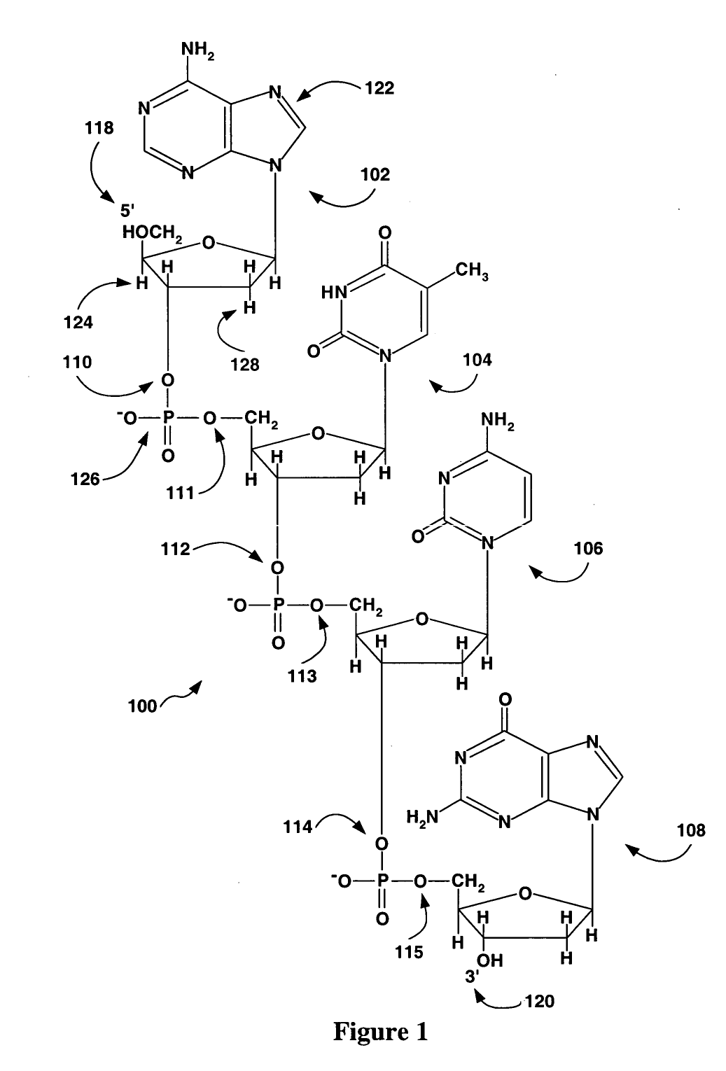

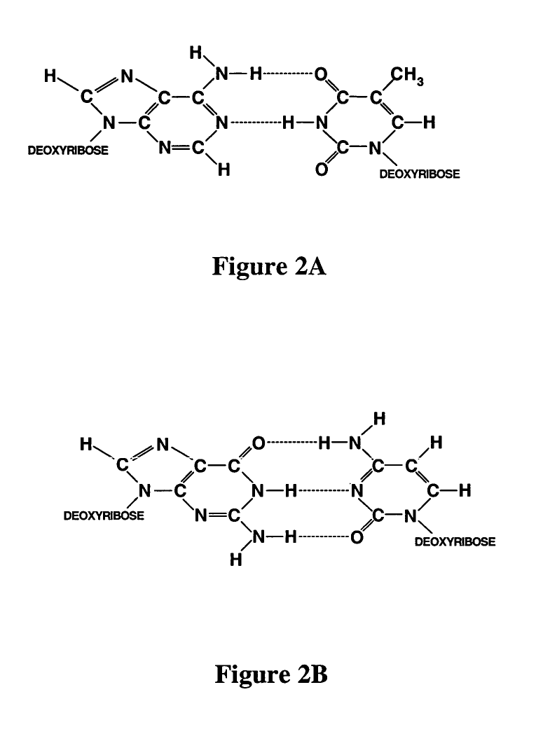

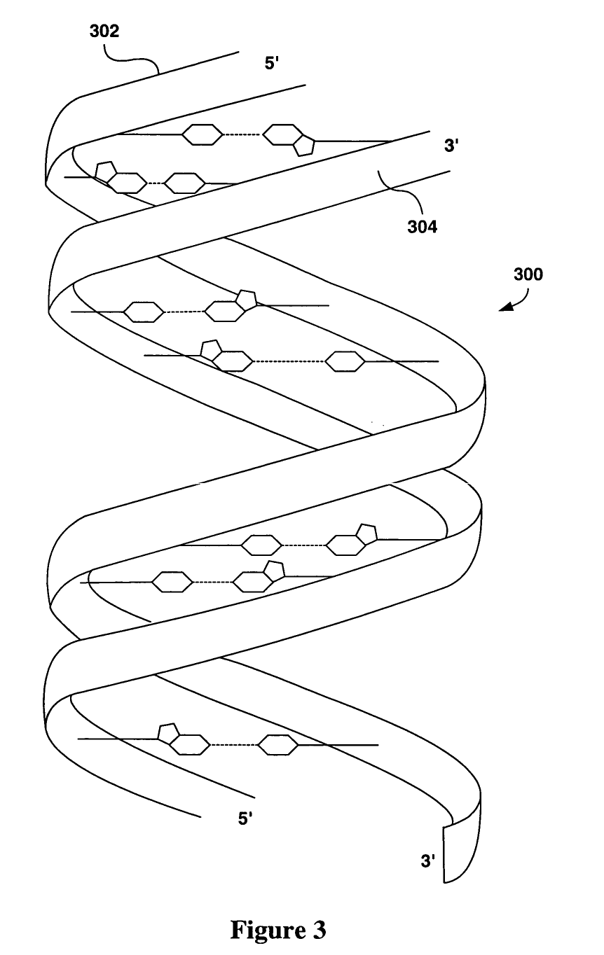

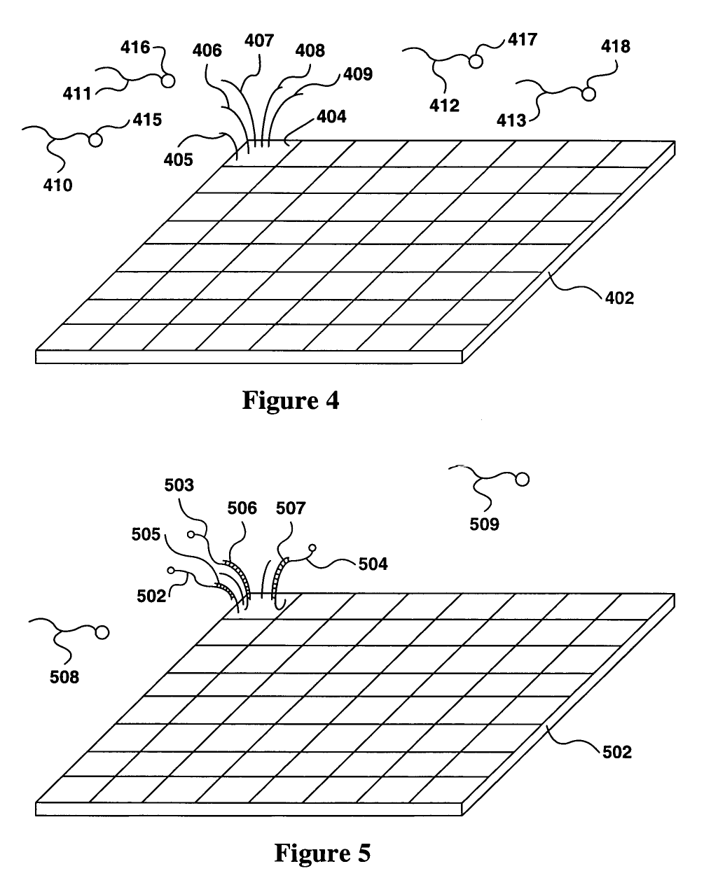

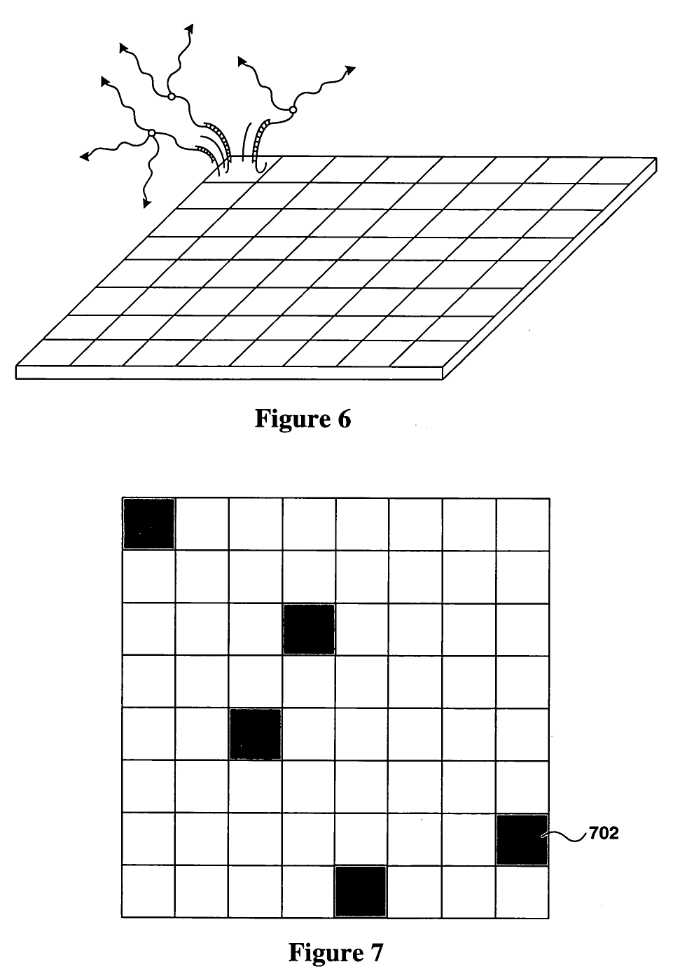

### DETAILED DESCRIPTION OF EMBODIMENTS OF THE INVENTION

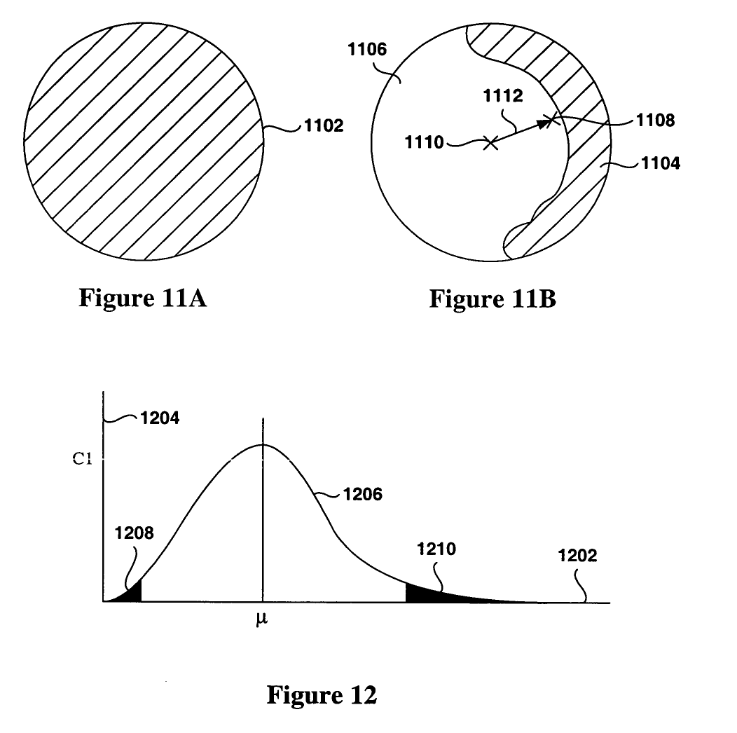

Various embodiments of the present invention are directed toward a method for quantifying random errors, sequence-dependent trends, and spatial-intensity trends in microarray data sets. The following discussion includes two subsections, a first subsection including additional information about molecular arrays, and a second subsection describing embodiments of the present invention with reference to FIGS. 11-15.

### Additional Information About Microarrays

A microarray may include any one-, two- or three-dimensional arrangement of addressable regions, or features, each bearing a particular chemical moiety or moieties, such as biopolymers, associated with that region. Any given microarray substrate may carry one, two, or four or more microarrays disposed on a front surface of the substrate. Depending upon the use, any or all of the microarrays may be the same or different from one another and each may contain multiple spots or features. A typical microarray may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the microarrays are formed by processes involving drop deposition of reagents, but may not be present when, for example, photolithographic microarray fabrication processes are used. When present, interfeature areas can be of various sizes and configurations.

Each microarray may cover an area of less than 100 cm², or even less than 50 cm², 10 cm² or 1 cm². In many embodiments, the substrate carrying the one or more microarrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With microarrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Microarrays can be fabricated using drop deposition from pulsejets of either polynucleotide precursor units (such as monomers) in the case of in situ fabrication, or the previously obtained polynucleotide. Such methods are described in detail in, for example, U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic microarray fabrication methods may be used. Interfeature areas need not be present particularly when the microarrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the microarray, and the microarray is then read. Reading of the microarray may be accomplished by illuminating the microarray and reading the location and intensity of resulting fluorescence at multiple regions on each feature of the microarray. For example, a scanner may be used for this purpose, which is similar to the AGILENT MICROARRAY SCANNER manufactured by Agilent Technologies, Palo Alto, Calif. Other suitable apparatus and methods are described in published U.S. patent applications 20030160183A1, 20020160369A1, 20040023224A1, and 20040021055A, as well as U.S. Pat. No. 6,406,849. However, microarrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, and elsewhere.

A result obtained from reading a microarray, followed by application of a method of the present invention, may be used in that form or may be further processed to generate a result such as that obtained by forming conclusions based on the pattern read from the microarray, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically tran-sporting that item or, in the case of data, physically transporting a medium carrying the data or communicating the data.

As pointed out above, microarray-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the microarray that would bind to soluble labeled antigens in a sample solution. Many other types of chemical assays may be facilitated by microarray technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for microarray-based analysis. A fundamental principle upon which microarrays are based is that of specific recognition, by probe molecules affixed to the microarray, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces an image comprising a rectilinear grid of pixels, with each pixel having a corresponding signal intensity. These signal intensities are processed by a microarray-data-processing program that analyzes data scanned from an microarray to produce experimental or diagnostic results which are stored in a computer-readable medium, transferred to an intercommunicating entity via electronic signals, printed in a human-readable format, or otherwise made available for further use. Microarray experiments can indicate precise gene-expression responses of organisms to drugs, other chemical and biological substances, environmental factors, and other effects. Microarray experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Embodiments of the Present Invention

In general, the intensity associated with a feature of a microarray is the sum of: (1) a first signal-intensity component produced by specifically bound target molecule labels; and (2) a second signal-intensity component, referred to as the “induced signal intensity,” which may be the product of a wide variety of background-intensity-producing sources, including noise produced by electronic and optical components of a microarray scanner, general non-specific reflection of light from the surface of the microarray during scanning, labeled target molecules non-specifically hybridized to feature probes or, in the case of radio-labeled target molecules, natural sources of background radiation, and various defects and contaminants on, and damage associated with, the surface of the microarray. Random appearing manufacturing defects, randomly distributed contaminants on the surface of the microarray, and noise are referred to as “random errors.”

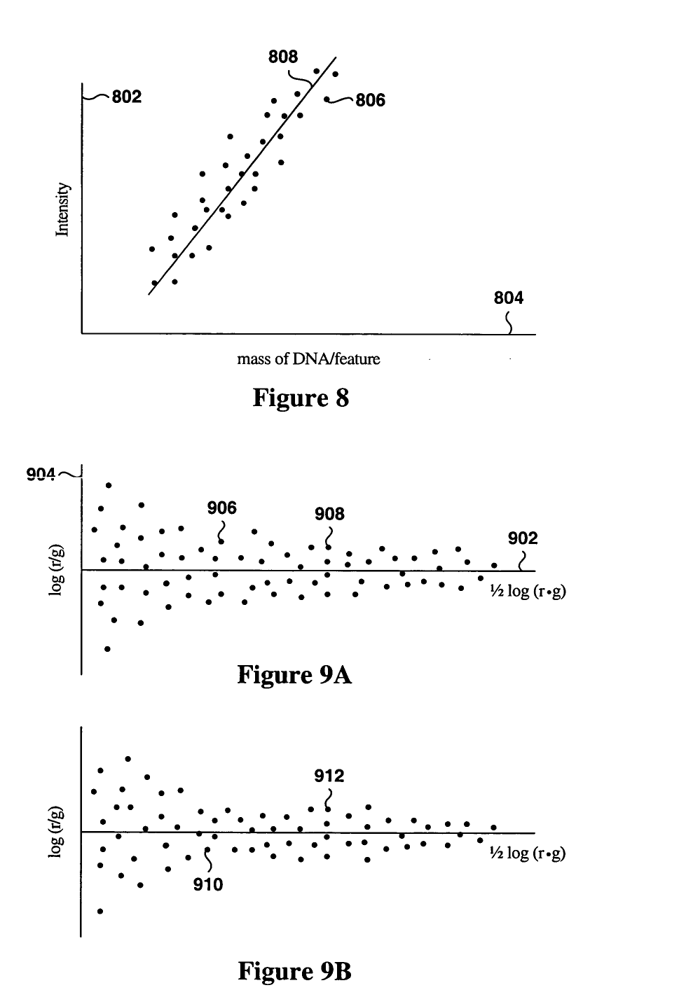

The second signal-intensity component may contain signal intensities emitted by probe molecules bound to a feature, which in turn, may be the result of weak intrinsic fluorescent properties, radiation used to stimulate emission from hybridized target molecule labels, or a contaminant bound to, or associated with, the probe molecules. The signals emitted by bound oligonucleotide probe molecules may be sequence dependent. For example, signal strengths produced by the four DNA nucleotide bases of oligonucleotide probes vary from a weakest signal produced by deoxy-adenosine, to intermediate strength signals produced by deoxy-thymidine and deoxy-guanosine, in that order of respective strength, to a strongest signal intensity produced by deoxy-cytosine. Therefore, oligonucleotide probes with a high proportion of deoxy-adenosine monomers, generally produce smaller second signal-intensity components, while oligonucleotide probes with a high proportion of deoxy-cytosine nucleotides generally produce larger second signal-intensity components. The strengths of the induced signals emitted by probe molecules may also be proportional to the mass of the probe molecules. FIG. 8 is a hypothetical plot of the relationship between induced signal intensities produced by unhybridized probes and the corresponding mass of probe molecules. In FIG. 8, vertical axis 802 corresponds to signal intensity level, and horizontal axis 804 corresponds to mass of the oligonucleotide probes bound to various features. In FIG. 8, each data point, such as data point 806, represents a probe molecule mass and corresponding feature intensity. The trend in the data points reveals that, as the nucleotide mass per feature is increased, the corresponding induced signal intensity level also increases, as indicated by the best-fit line 808.

Typically, second signal-intensity components are ignored in analysis of microarray data sets. However, second signal-intensity components can be large enough to influence the results of gene expression assays. For example, consider two hypothetical genes i and j for which expression levels in two hypothetical sample solutions, denoted by s and t, are measured. In a first hypothetical microarray hybridization assay, hypothetical sample solution s is prepared with red labeled target molecules, and hypothetical sample solution t is prepared with green-labeled target molecules. FIG. 9A shows a log ratio plot of a hypothetical hybridization assay for sample solutions s and t. In FIG. 9A, and in FIG. 9B described below, the horizontal axes, such as axis 902, correspond to

$$\frac{1}{2} log ( r \cdot g ) ,$$

and the vertical axes, such as axis 904, correspond to

$$log ( \frac{r}{g} )$$

for the red to green channel signal intensities r and g. In FIGS. 9A-B, each data point, such as data point 906, corresponds to the log ratio of red signal to green signal versus red signal for a feature of a microarray. The data points corresponding to features often form a cloud-like distribution about the horizontal axis 902. In FIG. 9A, genes i and j are identified by data points 906 and 908, respectively. Note that both data points exhibit positive log ratios, indicating that the expression levels for both genes is higher in sample solution s than in sample solution t.

Next, a microarray-based hybridization assay is carried out using sample solutions s′ and t′. Sample solution s′ includes the same target molecules as sample solution s, but sample solution s′ is prepared with green labels. Similarly, sample solution t′ includes the same target molecules as sample solution t, but sample solution t′ is prepared with red labels. This second microarray-based hybridization assay essentially exchanges the dyes used for the two sample solutions, and the pair of hypothetical microarray-based hybridization assays represents an example of a “dye-swap experiment.” FIG. 9B is a plot of the log ratios for the dye-swap microarray-based hybridization assay. In FIG. 9B, log ratios of genes i and j are identified by data points 910 and 912, respectively. Data point 910 represents an ideal result because gene i has a higher expression level in sample solution s′ than in sample solution t′, which is consistent with the result observed in the first hypothetical microarray hybridization assay. In other words, log(i,/i_{g})ₛₜ=−log(i_{g}/iᵣ)_{s′t′}. By contrast, data point 912 indicates that the expression level of gene j is higher in sample solution t′ than in sample solution s′, which contradicts the results of the first microarray hybridization assay. Gene j, described above with reference to FIGS. 9A-B is referred to as a “crossover” gene because it appears to be in high abundance in both sample solutions s and t′. The crossover event displayed in FIGS. 9A-B for gene j can be caused by a large second signal-intensity component.

Unfortunately, it may be difficult to precisely determine the sources of the second signal-intensity component of features in a microarray data set, particularly when the microarray contains contaminants and/or defects that produce background intensity gradients in the microarray data set. Typically, sequence dependent variations in signal intensities appear as systematic variations in signal intensities across a microarray surface and are referred to as “sequence-dependent trends.” However, if the sequence composition of the microarray probes are randomly distributed, then the sequence-dependent trends appear random when viewed spatially. The sequence-dependent trends appear systematic when viewed along any axis which correctly displays the systematic intensity trends of the second signal-intensity component. A spatial variation in signal intensities across a microarray surface, referred to as a “spatial-intensity trend,” may include both the sequence-dependent trends and contributions from other systematic background intensities. Features having signal-intensities within about 2 to 3 standard deviations of the negative control features, referred to as the “lowest-signal-intensity features,” can be used to identify the presence of a spatial-intensity trends.

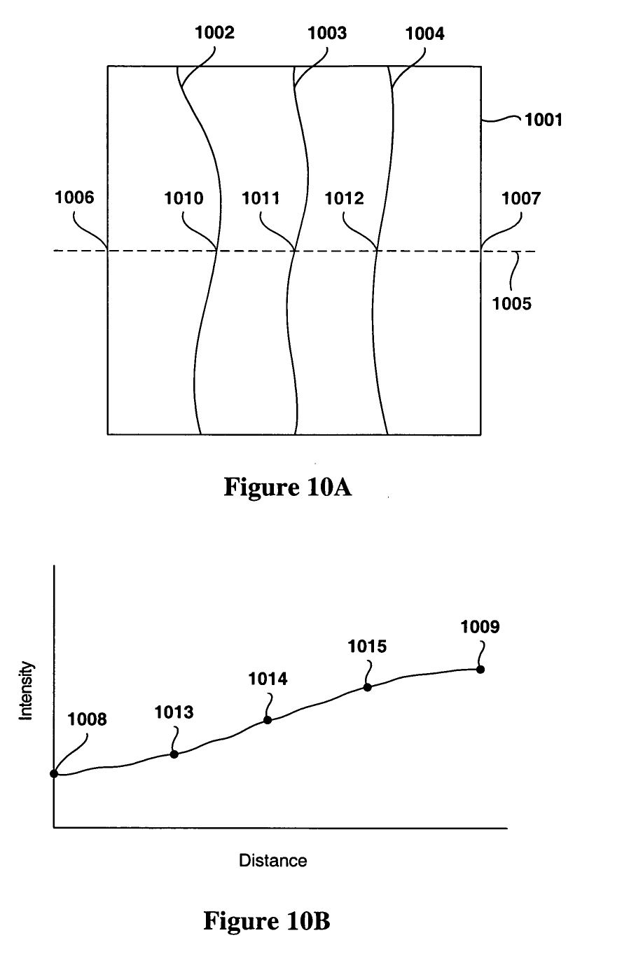

FIGS. 10A-B illustrates a spatial-intensity trend using a contour plot of the lowest-signal-intensity features for one channel of a microarray 1001. A contour line indicates a set of features all with nearly equal intensities, just as a contour line on topographic map indicates terrain at a particular elevation. In FIG. 10A, contour lines 1002-1004 identify lowest-signal-intensity features having these particular intensity values, increasing from left to right. FIG. 10B shows a graph of feature intensity values versus position along a horizontal line roughly bisecting the contour plot shown in FIG. 10A. The plot shown in FIG. 10B may be obtained by plotting the intensity values associated with features along bisecting line 1005 in FIG. 10A. In FIG. 10B, points 1008 and 1009 correspond to the points 1006 and 1007 in FIG. 10A, respectively. FIG. 10B is essentially a vertical cross-section, along line 1005, of the intensity map shown in FIG. 10A. The increasing signal trend represented by contour lines in FIG. 10A is readily observed as a slope of intensity values rising towards the right-hand side of the plot in FIG. 10B.

One of many possible embodiments of the present invention is directed to a method for quantifying random errors, sequence-dependent trends, and spatial-intensity trends present in multi-channel, microarray data sets. Random errors, sequence-dependent trends, and spatial-intensity trends present in a microarray data set can be quantified by computing an additive error, denoted by AddError, computed as follows:
AddError_{(C)}=\sqrt{m₁²·σ_{NegCtrl(C)}²+m₂²·DNF_{(C)}²·(Spatial RMS Filtered minus Fit)_{(C)}²}

where C=channel index of the microarray data set;

σ_{NegCtrl}²=variance of the inlier negative controls of the corresponding channel;

DNF=linear dye normalization factor;

Spatial RMS Filtered minus Fit=root mean square (“RMS”) of the intensities defining the surface fit for the channel;

m₁=negative-control variance multiplier; and

m₂=spatial-intensity trend multiplier.

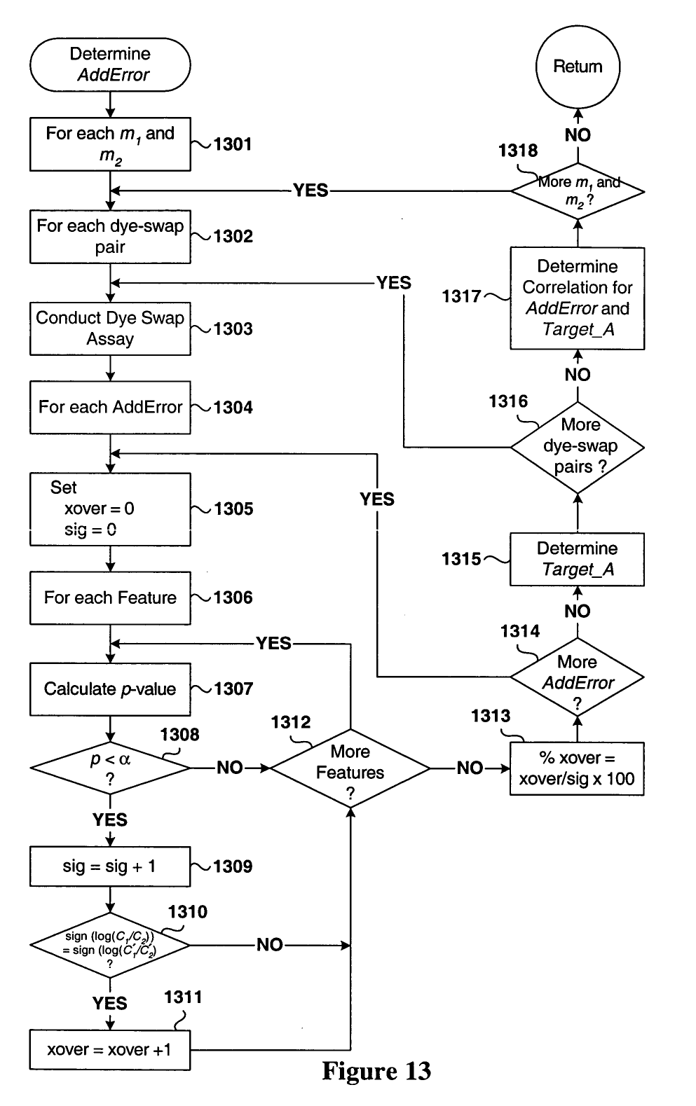

The additive error AddError is determined for each channel of a microarray data set. The variables σ_{NegCtrl}, DNF, and Spatial RMS Filtered minus Fit are determined for each channel of the microarray data set as described below, in greater detail, with reference to FIGS. 11 and 12. The constants m₁ and m₂ are determined separately, and also described below, in greater detail, with reference to FIGS. 13-16. Once the constants m₁ and m₂ have been determined as described below the constants may be used to determine the additive error AddError for subsequent microarray hybridization assays.

After dye normalization, the variance of negative control features, denoted by σ_{NegCrtl}², accounts for any random variation or noise in signal intensities that may be present in the microarray data set. Negative controls features are composed of bound probes designed not to specifically hybridize with any target molecules present in the sample solution, and therefore, typically produce low-feature-signal intensities. The variance a σ_{NegCrtl}² can be used to quantify random errors in signal intensities attributed to a microarray reader or the microarray surface, because negative controls are generally replicated in many feature locations across a microarray. Moreover, if the spatial-intensity trend is not removed from the microarray data set, as described in Agilent U.S. Patent Application entitled “Method and System for Quantifying and Removing Spatial-Intensity Trends in Microarray Data,” Attorney Docket No. 10040609, and filed on the same date as the present invention, which is incorporated by reference, the variance or σ_{NegCrtl}² can also be used to measure spatial dependence variability and any residual trend remaining even after the spatial-intensity trend has been removed. Prior to calculating the variance σ_{NegCrtl}², negative controls having non-uniform intensity distributions and negative controls having extreme signal intensities are identified and removed from consideration in the determination of the variance σ_{NegCrtl}². The negative control features having non-uniform distributions and negative control features having extreme signal intensities are referred to as “outlier negative controls,” and the remaining negative control features are referred to as “inlier negative controls.”

FIGS. 11A-B show both a negative-control feature having a uniform intensity distribution and a negative-control feature having a non-uniform intensity distribution. In FIG. 11A, the disc-shaped area of the image of a feature 1102 is shown with cross-hatching indicating a uniform distribution of low intensity values over the entire area of the image of the feature. By contrast, in FIG. 11B, a crescent-shaped portion 1104 of the area of a feature has low intensity values, while the remaining portion of a feature, 1106, has medium or high intensity values. The signal intensities within the feature shown in FIG. 11B are non-uniformly distributed. Non-uniform distribution of intensities can be detected in a number of different of ways. A statistical variance of pixel intensities within the area of the image of a feature can be computed, and features with pixel-intensity variances greater than a threshold variance can be considered to have non-uniform pixel-intensity distributions. For example, the threshold may be a function of biological or electronic noise, or noise on the microarray due to chemical or hybridization processes. Alternatively, as shown in FIG. 11B, a centroid for the pixel intensity distribution 1108 can be computed, and the location of the centroid compared to the location of the geometric center 1110 of the image of the feature. When the distance 1112 between the centroid of pixel-intensity and the geometric center is greater than a threshold distance value, the feature can be considered to have a non-uniform pixel-intensity distribution. Alternatively, both the mean and the median signal statistics can be computed and a feature can be considered to have a non-uniform pixel-intensity distribution if the difference between the mean signal and the median signal exceeds a threshold, or if the difference divided by either the mean or the median signal exceeds a threshold. Many other alternative techniques can be employed in order to classify negative control features having non-uniform pixel-intensity distributions.

The negative controls having signal intensities that are either extremely large or extremely small are not considered in determining the variance σ_{NegCrtl}², because these features tend to be much higher or lower than the normal distribution, and therefore may distort the magnitude of the variance σ_{NegCrtl}². FIG. 12 illustrates a hypothetical distribution of a set of negative-control feature intensities. In FIG. 12, horizontal axis 1202 corresponds to the negative-control feature signal intensities, vertical axis 1204 corresponds to the number of negative-control features, and the curve 1206 represents the distribution of negative-control feature signal intensities. The negative control features having extremely small or large signal intensity values are located in the upper or lower ends of the negative-control, feature-intensity distribution. For example, in FIG. 12, the negative-control features having extremely large and small signal intensities are identified by the shaded regions 1208 and 1210, and therefore, are not considered in determining the variance σ_{NegCrtl}².

After outlier negative controls have been identified, the channel variance of the microarray data set is determined using only the inlier negative control features, as follows:

$$\begin{matrix}{σ}_{Neg Crtl}^{2} = \frac{{\sum }_{i = 1}^{N} {( {I}_{Neg Ctrl , i} - {\overline{I}}_{Neg Crtl , i} )}^{2}}{N} \\ where {\overline{I}}_{Neg Ctrl} = \frac{{\sum }_{i = 1}^{N} {I}_{Neg Ctrl , i}}{N} ;\end{matrix}$$

N=number of inlier negative control features;
i=the inlier negative control feature index; and
I_{NegCrtl,i}=the signal intensity of the ith inlier negative control feature.
   Note that, if the spatial-intensity trend has been mostly removed, the variance σ_{NegCrtl}² may still capture any residual spatial-intensity trends. Note further that, if spatial-intensity trend has not been removed, the variance σ_{NegCrtl}² will capture both random errors and spatial-intensity trends. The magnitude of the spatial-intensity trend can be used to estimate the magnitude of the sequence-dependent trend.

Next, the linear-dye-normalization factor DNF given in the additive error AddError is determined. For each microarray hybridization assay, a number of features are dedicated to dye-normalization probes. Ideally, dye-normalization features should reveal nearly identical gene expression levels in each channel. However, the different labels used to measure gene expression levels have different signal emitting efficiencies. Therefore, dye-normalization features can be used to normalize the signal intensity data for each channel of microarray data set in order to remove non-biological variation that may be associated with the labels. The linear dye-normalization method assumes that dye bias is not intensity dependent, and therefore, takes a global approach to dye normalization. A linear dye-normalization factor is computed per microarray data set channel by setting the geometric mean of the signal intensity of the normalization features equation to 1000. The of the linear-dye-normalization factor is determined for each channel by:

$$DNF = \frac{1000}{{10}^{( \frac{1}{n} {\sum }_{i = 1}^{n} log {X}_{i} )}}$$

where Xᵢ=the signal intensity of the dye-normalization feature i; and
n=the number of dye-normalization features.

The Spatial RMS Filtered minus Fit term in the above equation for AddError is provided in detail and determined according to the method described in Agilent U.S. Patent Application entitled “Method and System for Quantifying and Removing Spatial-Intensity Trends in Microarray Data,” Attorey Docket No. 10040609, filed the same day as the present invention. The Spatial RMS Filtered minus Fit is a measure of the residual difference between the lowest-signal-intensity features (or highest-signal-intensity trends) and a surface characterizing the spatial-intensity trends. Before the microarray data processing dye-normalization step, the Spatial RMS Filtered minus Fit is determined and multiplied by the dye-normalization factor DNF to ensure that the Spatial RMS Filtered minus Fit units are identical to the variance σ_{NegCrtl}², units. Whether or not the background contribution has been removed from the microarray data set, the Spatial RMS Filtered minus Fit term includes the sequence-dependent trends in determining the additive error AddError. In other words, Spatial RMS Filtered minus Fit includes the sequence-dependent trend by assuming that the sequence-dependent trend is proportional, in magnitude, to the spatial-intensity trend.

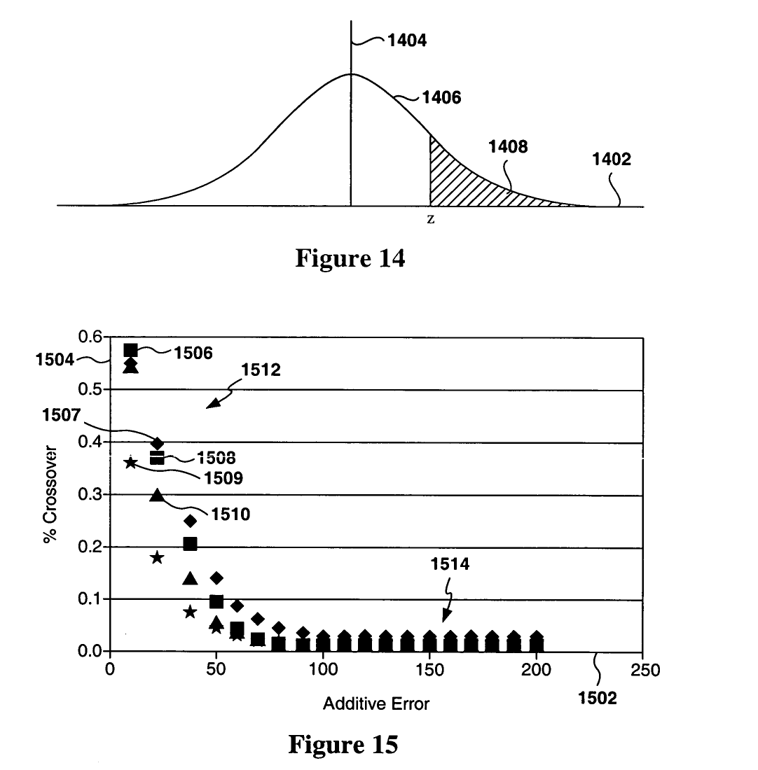

FIG. 13 is a control-flow diagram describing the method of one embodiment of the present invention for determining the constants m₁ and m₂ in the additive error AddError. This method is described with reference to FIGS. 13 and concurrent reference to FIGS. 14-16 that provide greater detail for many of the steps shown in FIG. 13.

The constants m₁ and m₂, once determined, as described below with reference to FIG. 13, are fixed constants that can be employed to determine an additive error AddError value for a number of different microarray hybridization assays. The constants m₁ and m₂ are determined heuristically. The outer for-loop, consisting of steps 1301-1318, repeats steps 1302-1318 for a number of different m₁ and m₂ constants. Intermediate for-loop, consisting of steps 1302-1316, repeats steps 1303-1316 for a mumber of different dye-swap microarray hybridizations assays. Initially, in step 1303, a dye-swap microarray hybridization assay is conducted using two identical microarrays. Intermediate for-loop, consisting of steps 1304-1316, repeats steps 1305-1316 for additive error AddError values ranging between min(AddError) and max(AddError) for a particular pair of constants m₁ and m₂. In step 1305, the variables “xover” and “sig” are each assigned the value “0.” The variables “xover”and “sig” count the number of crossover features and the number of significant features, respectively. Inner for-loop, consisting of steps 1306-1312, repreats steps 1307-1312 for each feature of a dye-swap microarray data set. In step 1307, for each feature, a p-value is calculated to determined which features are significant. The p-value is computed according to the following equation:

$$\begin{matrix}p ( z ) = 1 - erf ( z ) \\ where \\ erf ( z ) = \frac{1}{\sqrt{π}} {\int }_{- ∞}^{z} {ⅇ}^{- {t}^{2}} ⅆ t is the error function ; \\ z = \frac{{C}_{1 , i} - {C}_{2 , i}}{\sqrt{{AddError ( {C}_{1} )}^{2} + {AddError ( {C}_{2} )}^{2} + {( M ( {C}_{1} ) \cdot {C}_{1 , i} )}^{2} + {( M ( {C}_{2} ) \cdot {C}_{2 , i} )}^{2}}} ;\end{matrix}$$

C_{l,i}=C₁ channel intensity at feature i;

C_{2,i}=C₂ channel intensity at feature i;

AddError(C₁)=additive error determined, according to additive error AddError equation for the channel C₁;

AddError(C₂)=additive error determined, according to additive error AddError equation for the channel C₂;

M(C₁)=multiplicative error for the C₁ channel; and

M(C₂)=multiplicative error for the C₂ channel.

The p-value is the incomplete error function, which is also given by the equation:

$$p ( z ) = \frac{2}{\sqrt{π}} {\int }_{z}^{∞} {ⅇ}^{- {t}^{2}} ⅆ t$$

The p-value represents the area under the integrand

$$\frac{2 {ⅇ}^{- {t}^{2}}}{\sqrt{π}}$$

from z to infinity.

FIG. 14 illustrates the concept of a p-value. In FIG. 14, the horizontal axis 1402 corresponds to z values given above in the AddError equation, and vertical axis 1404 corresponds to the values of the integrand

$$\frac{2 {ⅇ}^{- {t}^{2}}}{\sqrt{π}} .$$

The curve 1406 represents the normal distribution given by the function

$$\frac{2 {ⅇ}^{- {t}^{2}}}{\sqrt{π}} .$$

The area under curve 1406 from negative infinity to positive infinity is equal to “1.” For a z, determined as described above in the AddError equation, the area of the hash-marked region 1408 represents the p-value.

The p-value is used to determine whether a conclusion based on a given measurement is likely to be true. A measurement that leads to a correct conclusion with high probability is called “significant.” Whether or not a measure is significant, based on its corresponding p-value, is determined by a user-supplied significance level referred to as α. For example, if the user specifies a significance level α equal to 0.01, then a measurement that gives a p-value less than a means that there is a greater than 99% (1-0.01=0.99) chance that a conclusion based on the measurement is correct.

In step 1308, if the p-value for a given feature is less than the user-specified significance level α, then, in step 1309, the variable “sig” is incremented by “1.” Next, in step 1310, if the sign of log(C₁/C₂) is identical to the sign of the dye-swap log (C₁/C₂), where C₁′ and C₂′ are the dye-swapped channels in the dye-swap hybridization assay, then, in step 1311, the variable “xover” is incremented by “1.” In steps 1308 and 1310, if the p-value is greater than the significance level α, or the sign of the log ratios are opposite, then control proceeds to step 1312. In step 1312, if more features are needed, then steps 1307-1311 are repeated. In step 1312, if there are no more features to consider, then, in step 1313, the percent of crossover genes is calculated as follows:

$$\% xover = \frac{xover}{sig} \times 100$$

Next, in step 1314, if no more additive error terms AddError are needed, then, in step 1315, the minimum crossover point is determined as described below with reference to FIG. 15.

FIG. 15 is a plot of the percent of crossover genes versus additive error AddError for a single hypothetical, dye-swap microarray hybridization assay. In FIG. 15, the horizontal axis 1502 corresponds to the additive error AddError, and the vertical axis 1504 corresponds to the percent of gene crossover in the dye-swap microarray hybridization assay. In FIG. 15, each data point, such as data point 1506, represents the percentage of crossover genes versus additive error for a particular pair of constants m₁ and m₂. Four different data sets identified by four different data point shapes 1507-1510 are plotted in FIG. 15. The four different data sets represent the percentage of crossover genes versus additive error for different multiplicative constants M(C₁) and M(C₂) given above in z in the equation. Each set of data points shown in FIG. 15 is generated for a given microarray data set by repeated application of steps 1303-1313 in FIG. 13. All four sets of data points displayed in FIG. 15 reveal a similar pattern of steep descent, identified by region 1512, followed by a flat region 1514, as the additive error AddError increases in magnitude. The optimal values for the constants m₁ and m₂ can be approximated by locating a point where the set of data points transition from the steep descent region 1512 to the flat region 1514, which is identified by the additive error AddError point 1516 and is referred to as Target_A. Target_A 1516 identifies the additive error AddError value that minimizes crossover for a particular set of data points, while minimally decreasing the total number of features called significant.

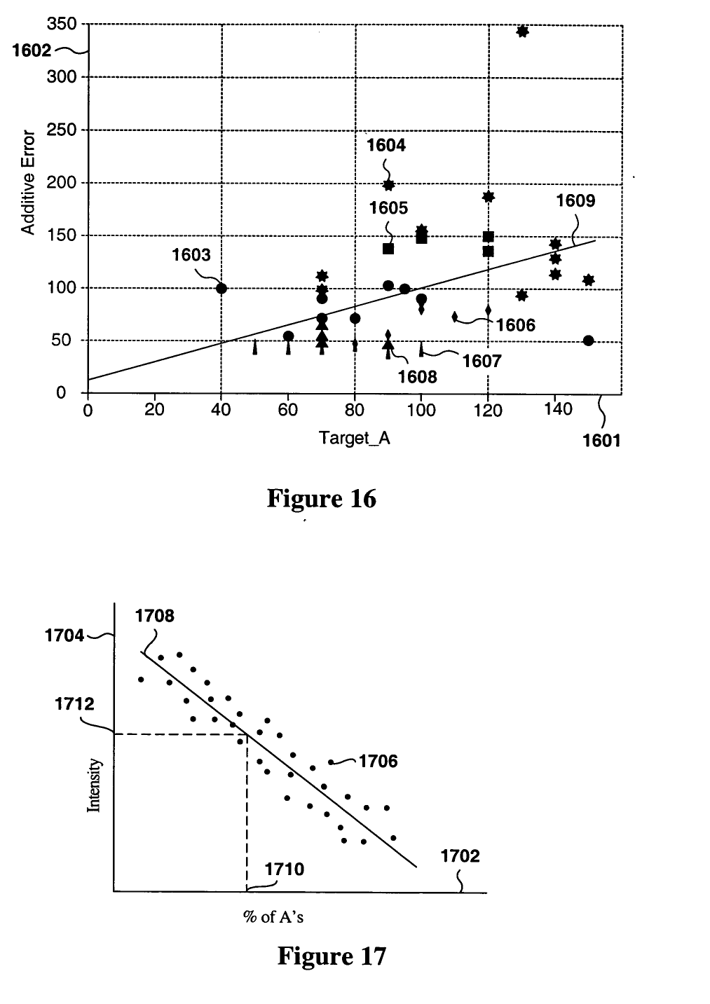

In step 1316, if more dye-swap microarrays are needed, then steps 1303-1315 are repeated. In step 1316, if no more dye-swap microarrays are needed, then in step 1317, the correlation between the additive error AddError and the Target_A's is measured for a particular pair of constants m₁ and m₂ as described below in relation to FIG. 16.

FIG. 16 is a plot of the additive error AddError versus Target_A values for a particular pair of constants m₁ and m₂. In FIG. 16, horizontal axis 1601 corresponds to the Target_A values, and vertical axis 1602 corresponds to the additive error AddError for the same pair of constants m₁ and m₂. Each data point, such as data point 1603, represents the additive error AddError and corresponding Target_A for one of 41 different dye-swap microarray hybridization assays. The additive error AddError for all data points is computed using the same m₁ and m₂ constants. The six different data point shapes, such as data points 1603-1608, represent six different kinds of microarray assays. For example, round data points, represented by data point 1603, correspond to using double-density microarrays, and star-shaped data points, such as data point 1604, represent using 8-pack microarrays. A best-fit trend line 1609 is determined using the entire set of data points shown in FIG. 16. The correlation for the entire set of data points is used to measure the strength of the linear relationship between the additive error AddError and the Target_A values for a particular pair of constants m₁ and m₂. One of many possible methods for measuring the correlation is given by:

$$ρ = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

where n=number of data points;
xᵢ=Target_Aᵢ;
\overline{x}=mean Target_A;
yᵢ=AddErrorᵢ; and
\overline{y}=mean AddError.
   The correlation ρ values ranges from −1 to 1. A correlation ρ value close to “0” means the additive error AddError and corresponding Target_A values are uncorrelated. A correlation ρ value close to 1 or −1 means there is a linear relationship between the additive error AddError and corresponding Target_A values. A correlation value ρ is determined for each pair of constants m₁ and m₂ using a number of different dye-swap microarray hybridization assays.

In step 1318, if more constants m₁ and m₂ are needed, then steps 1302-1316 are repeated. The pair of constants m₁ and m₂ that provides the best correlation ρ (i.e., have correlation ρ closest to 1 or −1) are considered to be optimal constants, and can be used to calculate the additive error AddError for a variety of microarrays.

Although the present invention has been described in terms of a particular embodiment, it is not intended that the invention be limited to this embodiment. Modifications within the spirit of the invention will be apparent to those skilled in the art.

For example, in an alternate embodiment, the negative-control variance parameter ml can be assigned the value “1,” because the variance σ_{NegCrtl}² which represents the random noise of the microarray, accounts for any random variation in signal intensities that may be present in the microarray data set. Therefore, in FIG. 13, for each microarray in step 1301, only the spatial-intensity trend multiplier m₂ needs to be varied over a predetermined range of values in steps 1304-1313.

In alternate embodiments, rather than employing identical negative-control, oligonucleotide sequences, a variety of oligonucleotide probe sequences can be used. If the negative-control oligonucleotide sequence composition distribution is similar to the distribution of non-negative control feature oligonucleotide probes, then the additive error AddError can be determined using only the variance σ_{NegCrtl}². Note that, the negative controls sequences are not required to match the non-negative-control sequences in determining similarity between sequence composition distributions. Only the distributions having similar sequence parameters, such as percentage of deoxy-adensosine, deoxy-guanosine, deoxy-cytosine, or deoxy-thymidine are needed. For example, the negative control features and the non-negative control features having probe sequences with similar percentage of deoxy-adenosine are considered to have similar nucleotide sequence composition distributions. The variance σ_{NegCrtl}² alone can be used to determine additive error AddError, because negative-control features: (1) are spatially distributed across the microarray; (2) measure random errors; and (3) intrinsically include any sequence-dependent trend. Furthermore, in this same embodiment, a functional relationship between the intensity of the second signal-intensity component (i.e., background) and a metric for the negative-control oligonucleotide sequence composition can be used to correct for sequence-dependent trends in non-negative-control features. In alternate embodiments, the metric may depend on the composition of the negative-control oligonucleotide probe sequence composition. Consider for the sake of simplicity, a metric that depends on the percentage of deoxy-adenosine present in oligonucleotide probe sequences. FIG. 17 is a plot of intensity versus percentage of deoxy-adenosine present in the negative-control oligonucleotide probes of a hypothetical microarray having different nucleotide sequence compositions. In FIG. 17, horizontal axis 1702 corresponds to the percentage of deoxy-adenosine present in the negative-control features, and vertical axis 1704 corresponds to the negative-control feature intensity. Data points represent the percentage of deoxy-adenosine present and the corresponding negative-control feature intensity, such as data point 1706. Note that, as the percentage of deoxy-adenosine present in the negative-control oligonucleotide probe sequences increases, the intensity decreases, as indicated by the negative sloping best-fit trend line 1708. The negative-control, best-fit trend line can be used to determine the sequence-dependent contribution to the second signal-intensity component for non-negative control features. Therefore, in addition to determining the sequence-dependent contribution, the signal emitted from a non-negative-control feature can be used to correct for sequence-dependent contributions present in non-negative-control features by subtracting the sequence-dependent contribution from the feature intensity. The linear relationship identified in FIG. 17 by the negatively sloping best-fit trend line 1708 can be used to correct for sequence-dependent contributions in the signals emitted by non-negative control features of the hypothetical microarray data set. In particular, the sequence-dependent contribution to the signal emitted by all non-negative-control features having a percentage of A's 1710 can be corrected by subtracting intensity 1712. Therefore, the signal associated with every non-negative-control feature could have a different amount of signal subtracted. In alternate embodiments, for example 8-pack microarrays, only the variance of the inlier negative controls is used to determine the additive error AddError. In alternate embodiments, rather than employing the Spatial RMS Filtered minus Fit in the equation for the additive error AddError, other metrics, such as the standard deviation of surface fit for the lowest-signal-intensity features for each channel, can be used to characterize the spatial-intensity trend. In alternate embodiments, additional terms that characterize the sequence-dependent trends and others signal intensity related anomalies could be included in the additive error AddError.

The foregoing description, for purposes of explanation, used specific nomenclature to provide a thorough understanding of the invention. However, it will be apparent to one skilled in the art that the specific details are not required in order to practice the invention. The foregoing description of specific embodiments of the present invention are presented for purposes of illustration and description. They are not intended to be exhaustive or to limit the invention to the precise forms disclosed. Obviously many modifications and variations are possible in view of the above teachings. The embodiments are shown and described in order to best explain the of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for quantifying background intensity trends in a microarray data set having one or more channels, the method comprising:
determining a random error contribution to background intensities in the microarray data set;
determining a spatial-intensity trend for each channel;
determining a sequence-dependent trend for each channel; and
determining an additive error for each channel of the data set from the determined random error contribution, spatial-intensity trend contribution, and sequence-dependent trend contribution.

2. The method of claim 1 wherein the magnitude of the spatial-intensity trend is proportional to the magnitude of the sequence-dependent trend.

3. The method of claim 1 wherein determining the random error contribution further includes:
selecting negative-control features from the microarray data set as an initial subset;
removing, from the initial subset negative-control, features having non-uniform intensity distributions;
removing, from the initial subset, negative-control features having extremely large or extremely small signal intensities compared to a mean signal intensity and a width of a negative-control intensity distribution; and
determining a variance of the negative-control-feature signal intensities based on negative-control features remaining in the initial subset.

4. The method of claim 1 wherein determining the spatial-intensity trend contribution further includes:
determining a linear dye-normalization factor based on a geometric mean of feature intensities; and
measuring a residual difference between the lowest-signal-intensity features or highest-signal-intensity trends and the spatial-intensity trends.

5. The method of claim 4 wherein determining the additive error further includes:
determining an optimal random error multiplier and a spatial-intensity trend multiplier;
summing of the random error contribution multiplied by the optimal random error
multiplier and the spatial-intensity trend contribution multiplied by the optimal spatial-intensity trend multiplier in quadrature; and
taking a square root of the sum.

6. The method of claim 5 wherein the optimal random error multiplier and the optimal spatial-intensity trend multiplier are determined by:
considering a number of different constant values for the random error multiplier and the spatial-intensity trend multiplier;
conducting one or more dye-swap microarray hybridization assays; and
determining a minimum percent crossover versus additive error for each dye-swap microarray hybridization assay.

7. The method of claim 6 wherein determining the percent crossover further includes determining significant features.

8. The method of claim 6 wherein determining the optimal random error multiplier and the optimal spatial-intensity trend multiplier further includes determining a minimum percentage of crossover value with minimal effect on a total number of significant features for each dye-swap microarray hybridization assay.

9. The method of claim 8 wherein determining the optimal random error multiplier and optimal spatial-intensity trend multiplier further includes determining a correlation between the additive error values and corresponding minimum crossover value for a number of different dye-swap microarray hybridization assays.

10. The method of claim 9 wherein the additive error values are determined for each dye-swap microarray hybridization assay using the same pair of random error and spatial-intensity trend multiplier constants.

11. A method for quantifying and correcting background intensity trends in a microarray data set having one or more channels, the method comprising:
determining a random error for each channel of the microarray data set;
determining an additive error for each channel of the microarray data set from the determined random error; and
correcting a sequence-dependent trend in the data set.

12. The method of claim 11 wherein determining the random error contribution further includes:
selecting negative-control features composed of varying oligonucleotide sequences from the microarray data set as an initial subset;
removing, from the initial subset, negative-control features having non-uniform intensity distributions;
removing, from the initial subset, negative-control features having extremely large or extremely small signal intensities compared to a mean signal intensity and a width of a negative-control intensity distribution; and
determining the variance of the negative-control-feature signal intensities based on negative-control features remaining in the initial subset.

13. The method of claim 11 wherein correcting the sequence-dependent trend in the data set further includes:
determining a function that characterizes sequence-dependent intensities in the negative-control features;
determining the sequence-dependent intensity for non-negative-control features based on the function that characterizes sequence-dependent intensities of the negative-control features; and
subtracting the sequence-dependent intensities from intensities for each non-negative-control feature based on the function values that characterizes sequence-dependent intensities of the negative-control features.

14. A representation of the additive error, produced using the method of claim 1, that is maintained for subsequent analysis by one of:
storing the representation of the additive error of the data set in a computer-readable medium; and
transferring the representation of the additive error of the data set to an intercommunicating entity via electronic signals.

15. Results produced by a microarray data processing program employing the method of claim 1 stored in a computer-readable medium.

16. Results produced by a microarray data processing program employing the method of claim 1 printed in a human-readable format.

17. Results produced by a microarray data processing program employing the method of claim 1 transferred to an intercommunicating entity via electronic signals.

18. A method comprising communicating to a remote location an additive error obtained by a method of claim 1.

19. A method comprising receiving data produced by using the method of claim 1.

20. A system for determining spatial-intensity trends in microarray data, the system comprising:
a computer processor;
a communications medium by which microarray data are received by the microarray-data processing system; and
a program, stored in the one or more memory components and executed by the computer processor that determines a random error contribution to the background intensities; determines a spatial-intensity trend for each channel; determines a sequence-dependent trend for each channel; and determines an additive error for each channel of the data set from the determined random error, spatial-intensity trend, and sequence-dependent trend.

21. A computer readable medium encoding instructions that implement the method of claim 1.
